(19) United States
(12) Patent Application Publication
(10) Pub. No.: US 2022/0336048 A1
(43) Pub. Date: Oct. 20, 2022

(54) METHODS FOR NEIGHBORHOOD PHENOMAPPING FOR CLINICAL TRIALS FOR INDIVIDUALIZED INFERENCE

(71) Applicant: Yale University, New Haven, CT (US)

(72) Inventors: Evangelos Oikonomou, New Haven, CT (US); Rohan Khera, Fairfield, CT (US)

(21) Appl. No.: 17/720,068

(22) Filed: Apr. 13, 2022

Related U.S. Application Data
(60) Provisional application No. 63/177,117, filed on Apr. 20, 2021.

(51) Int. Cl. G16B 20/40; G16B 40/00; G16H 10/20; G16H 50/20

(52) U.S. Cl. CPC G16B 20/40; G16B 40/00; G16H 10/20; G16H 50/20

(57) ABSTRACT

One aspect of the invention provides a method for phenotype mapping clinical trial participants. The method includes: receiving a set of data corresponding to a plurality of characteristics for a plurality of individual participants; classifying each individual patient based on the plurality of characteristics and according to a dissimilarity index; determining a dissimilarity value for each individual patient with respect to each of the remaining individual patients; and generating a phenotype neighborhood map comprising graphical representations for each individual patient. A distance between one individual patient and another individual patient is according to the dissimilarity value determined for the one patient with respect to the other individual patient.

### CROSS-REFERENCE TO RELATED APPLICATION(S)

This application claims the benefit of priority under 35 U.S.C. § 119(e) to U.S. Patent Application Ser. No. 63/177,117, filed Apr. 20, 2021. The entire content of this application is hereby incorporated by reference herein.

### BACKGROUND OF THE INVENTION

Randomized clinical trials (RCT) represent the highest level of evidence as they experimentally uncover effective diagnostic and therapeutic strategies. The key principle of an RCT is the unbiased allocation of an intervention to a group of individuals who are well characterized, with careful and systematic assessment of their subsequent clinical outcomes, compared against individuals not receiving that intervention. A carefully conducted, large randomized trial is often resource intensive requiring millions of dollars in recruitment, testing, and follow up. However, the inference from such well-conducted experiments is currently limited to their top-line results or assessments of a few major subgroups. The wealth of data collected in a clinical trial can be leveraged to better inform care and outcomes.

For example, nearly 200 million people globally suffer from coronary artery disease (CAD), one-half of whom initially present with chest pain. The optimal non-invasive diagnostic strategy for chest pain in patients with suspected stable CAD is clinically important to define, yet remains uncertain. PROMISE (PROspective Multicenter Imaging Study for Evaluation of Chest Pain) recently demonstrated that anatomical imaging has comparable outcomes to stress testing and may improve long-term outcomes when used in addition to standard of care including stress testing. This allowed computed tomography coronary angiography (CTCA) to gain traction as an alternative to functional imaging. However, the choice between these two strategies remains arbitrary, despite over 14,000 randomized individuals across large, well-conducted trials. This clinical equipoise is evident in the recent European Society of Cardiology (ESC) guidelines that assign a Class I recommendation to both CTCA and non-invasive functional testing as appropriate initial tests to diagnose CAD in symptomatic patients.

The PROMISE trial remains the largest randomized controlled trial to have compared CTCA with functional testing in low-risk symptomatic patients with stable chest pain and included 10,003 individuals followed for a median 25 months. However, subsequent analyses have revealed evidence of heterogeneity across broad subgroups, with women compared with men, and patients with diabetes compared with those without diabetes experiencing fewer adverse cardiovascular events with anatomical testing than with functional testing.

Nevertheless, broad subgroup assessments do not account for large variation in demographic and clinical features within such subgroups. However, there are no tools that support individualization of the expected benefit of anatomical and functional imaging based on each patient's unique phenotype, which is essential for shared decision-making.

Another set of trials for the drug canagliflozin, the Canagliflozin Cardiovascular Assessment Study (CANVAS), demonstrated benefit from the drug in preventing cardiovascular adverse events among patients with type 2 diabetes mellitus. Patients with diabetes are at an elevated risk of adverse cardiovascular outcomes. However, these CANVAS trials needed to include over 10,000 individuals followed for over 3 years to demonstrate the benefit. This represents a challenge for bringing treatments to market and is inefficient as a subset of the population may derive a majority of the benefit, and enrollment of those individuals in trials would make the trials faster and more cost-effective.

### SUMMARY OF THE INVENTION

One aspect of the invention provides a method for phenotype mapping clinical trial participants. The method includes: receiving a set of data corresponding to a plurality of characteristics for a plurality of individual participants; classifying each individual patient based on the plurality of characteristics and according to a dissimilarity index; determining a dissimilarity value for each individual patient with respect to each of the remaining individual patients; and generating a phenotype neighborhood map comprising graphical representations for each individual patient. A distance between one individual patient and another individual patient is according to the dissimilarity value determined for the one patient with respect to the other individual patient.

This aspect of the invention can have a variety of embodiments. The method can further include grouping each individual patient into a neighborhood based on a phenotype similarity threshold and the determined dissimilarity values.

The plurality of characteristics can include demographics, anthropometrics, health condition risk factors, laboratory measurements, medications, health condition symptoms, clinical risk scores, imaging or other medical data, or a combination thereof.

The method can further include: identifying a treatment to be administered to the plurality of individual patients; selecting a set of characteristics from the plurality of characteristics; and determining a heterogeneity level in effects from the treatment on a subset of individual patients sharing the selected set of characteristics. The method can further include: identifying a plurality of characteristics for an individual apart from the individual trial participants; and determining a treatment outcome for the administered treatment and for the individual based on the determined heterogeneity level. The treatment to be administered can include a medication, procedural or surgical intervention, nutritional supplement, diagnostic or therapeutic strategy, or a combination thereof.

The method can further include: identifying a treatment to be administered to the plurality of individual patients; and training a machine-learning algorithm to identify associations above a predefined threshold between one or more of the plurality of characteristics and a patient result of the administered treatment. The machine-learning algorithm can be an extreme gradient boosting algorithm. The method can further include: retraining the machine-learning algorithm by selecting a different set of characteristics; and identifying associations between the different set of characteristics and the patient result of the administered treatment.

The phenotype neighborhood map can include graphical representations.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts an Alluvial diagram of diagnostic testing in PROMISE. Panel (A): Among 10 003 participants randomized to anatomical vs. functional testing in the PROMISE trial, a total of 4834 vs. 4734 individuals underwent an anatomical vs. functional test as their initial investigation (and were included in this study), with 402 patients receiving no testing and the remaining 29 undergoing invasive coronary angiography as the initial diagnostic test. CTCA, computed tomography coronary angiography; ECG, electrocardiography; PROMISE, PROspective Multicenter Imaging Study for Evaluation of Chest Pain.

FIG. 2 depicts phenomapping the patient with chest pain in PROMISE. We present a manifold embedding of the baseline phenotypic variance seen in the PROMISE chest pain population based on 57 pre-randomization phenotypic traits. Panel (A): Labelling of the phenomap based on the treatment allocation reveals homogeneous distribution of the two strategies in the topological space, consistent with the random allocation to the two groups. Panel (B): In contrast, baseline phenotypic traits, such as the pooled cohort equation-derived 10-year ASCVD score were heterogeneously distributed, suggestive of clustering along a spectrum of baseline risk phenotypes. Panels (C and D): Labelling of the phenomaps with the neighborhood-derived individualized risk estimates demonstrated distinct topological neighborhoods favoring anatomical imaging or functional testing based on the observed risk in PROMISE. ASCVD, atherosclerotic cardiovascular disease; PROMISE, Prospective Multicenter Imaging Study for Evaluation of Chest Pain.

FIG. 3 depicts an example of patient phenomapping for personalized risk assessment. Phenomapping of three PROMISE study participants, all 59-year-old women with a history of diabetes, hypertension who presented with atypical chest pain and a pre-test Diamond-Forrester score of 20%. Phenomapping revealed that despite the above similarities, the patients were located in spatially distinct areas of the phenomap when accounting for the multitude of their phenotypic traits (Panel (A)). Neighborhood-specific analysis further revealed differential benefit with anatomical vs. functional imaging for each one of these patients (Panels (B-D)). aHR, adjusted hazard ratio; ASA, aspirin; BMI, body mass index; CCB, calcium channel blocker; CI, confidence interval; HDL, high-density lipoprotein; PROMISE, PROspective Multicenter Imaging Study for Evaluation of Chest Pain.

FIG. 4 depicts development of a decision support tool to predict individualized benefit from anatomical vs. functional imaging in chest pain investigation. Panel (A): In a randomly selected sample of the PROMISE population, we trained an extreme gradient boosting tree to predict the phenomap-derived individualized risk with anatomical vs. functional imaging. We identified the most important input features based on the SHAP (Shapley Additive exPlanations) values and selected the top 12 predictors (all with feature importance of 0.03 or higher) to create an easy-to-use clinical support tool, named ASSIST©. Panel (B): To offer some insight into each variable contribution, we used a SHAP summary plot, in which the y-axis represents the variables in descending order of importance and the x-axis indicates the change in prediction. The gradient color denotes the original value for that variable (for instance, for Booleans such as hypertension or diabetes it only takes two colors, whereas for continuous variables it contains the whole spectrum), with each point representing an individual from the original training set. Negative SHAP values (x-axis) indicate improved outcomes with anatomical imaging (as seen among individuals with hypertension and diabetes) whereas positive values indicate improved outcomes with functional imaging. Panels (C and D): Notably, ASSIST© predictions were independent of the random assignment to the anatomical or functional testing group in both the training and testing sets of PROMISE. ASSIST, Anatomical vs. Stress teSting decIsion Support Tool; PROMISE, PROspective Multicenter Imaging Study for Evaluation of Chest Pain; SHAP, Shapley Additive exPlanations.

FIG. 5 depicts validation and performance of ASSIST in PROMISE. Application of the ASSIST tool in both the training and testing (validation) set of PROMISE demonstrated that concordance (vs. disagreement) between the ASSIST-proposed best initial diagnostic strategy and a patient random allocation to functional or anatomical imaging was associated with an approximate two-fold reduction in the risk of the study primary composite endpoint (Panels (A-C)), as well as a composite endpoint of all-cause mortality and non-fatal myocardial infarction (DandE). ASSIST, Anatomical vs. Stress teSting decIsion Support Tool; PROMISE, PROspective Multicenter Imaging Study for Evaluation of Chest Pain.

FIG. 6 demonstrates the application of phenomapping to the CANVAS trials. The tool INSIGHT developed in the CANVAS trial was applied to the CANVAS-R trial. The left panel demonstrates that the tool did not pre-select individuals based on their treatment assignment when applied to CANVAS-R, and therefore, randomization is demonstrably maintained across neighborhoods. On the right, in the CANVAS-R trial, INSIGHT identified a subset of the population that derived a majority of the benefit (middle plot), compared with those that INSIGHT did not suggest would derive a large benefit (right plot), with a significant statistical interaction (p=0.04). On the bottom, statistical interactions for singular subgroups age, sex, and a history of coronary artery disease, or a history of heart failure, are presented for comparison, and were all not significant, suggesting that phenomapping-derive precision therapeutics enhanced benefit identification that were not based on simple phenotypic groups.

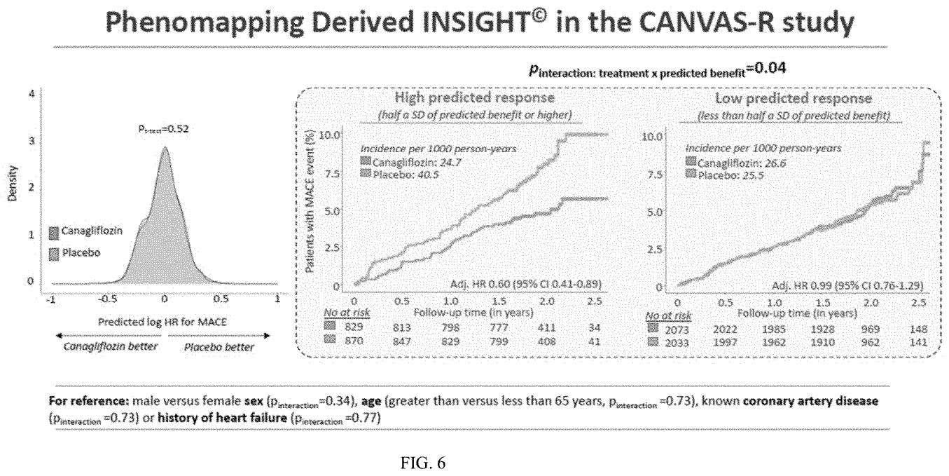

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Methods for neighborhood phenomapping clinical trial populations are described herein. A trial population can be transformed into a series of local experiments. Identification of a homogenous subset of patients enrolled in the trial can occur based on similarity of patient features before receiving the intervention. Since such subgroups are not defined by the actual intervention received, the intervention allocation and the inference of the treatment effect is unbiased. These data experiments embedded within the trial can provide novel insights about the effects of the intervention being tested in a trial, going beyond the reliance on top-line results.

The methods described herein combines concepts from the fields of machine learning (e.g., extreme gradient boosting algorithms) and visualization of multi-dimensional datasets (such as uniform manifold approximation and projection and neighborhood distance metrics) to uncover hidden heterogeneity in clinical trial data. An algorithm can iterate a neighborhood-specific analysis in the phenotypic neighborhood for each original patient included in the trial, thus producing n local experiments and enabling individualized prediction estimates. These methods can maintain the random assignment of all trial patients, while also enabling personalized risk estimates for prospective patients through their projection to the original trial risk phenomap, or through simplified machine-learning-derived risk tools directly derived from such phenomaps.

The methods described herein provide a multitude of benefits. For example, the methods allow for identifying how the results of an RCT affect a given individual based on that individual's set of characteristics. This is in contrast to the current approach of focusing on the average effect across people observed in the trial.

The conceptual framework retains the integrity of the trial design while defining heterogeneity of the effects of the intervention. This is accomplished by embedding local experiments within the trial population and defining each individual on a multitude of features, while ensuring that the intervention is allocated in an unbiased manner. This ensures that the findings are robust and unbiased and, therefore, can be translated to individuals outside the trial.

Machine learning can be applied to clinical trial populations, which can detect complex associations between patients, while also permitting flexibility to different trial designs and data sources. The application is not limited by the number or nature of features and can incorporate any or all information captured for individuals. The approach can include structured data (like comorbidities, vital signs, laboratory values, and baseline medications), but can also include unstructured data (notes, medical text), waveform data (such as electrocardiography), and medical imaging data to define these associations. Thus, the methods can be adapted for scalability to data with any structure and size.

Visualization techniques can ensure that the results can be interpretable, which can increase the ease of adoption. For example, multidimensional representation of trial participants can be represented in a 2D format, which can define participant features, proximity to other participants, response to therapy, and the like.

The minimum data necessary to provide information on precision effects can be identified. This identification can occur through dimensionality reduction and feature selection approaches that employ machine learning. Thus, the burden of data collection to define a person-specific intervention recommendation can be reduced.

The methods described herein can also result in simple clinical tools/algorithms that can be validated and generalized to large populations, integrating into the electronic health record for their prospective validation and clinical use. Each of these algorithms defined using different RCTs are themselves unique and represent independent intellectual contributions. Further, the methods described herein can be modelled for any of the clinical outcomes, with the ability to explicitly identify efficacy, safety, or net-benefit assessments.

### Experiment 1

In this study, we developed a method that evaluates the phenotypic diversity of patients presenting with stable chest pain as well as their optimal non-invasive testing strategy based on each patient's unique set of pre-randomization characteristics, and subsequent outcomes, using individual patient data from a major clinical trial investigating the clinical value of anatomical testing in the evaluation of chest pain.

Methods

### Data Source

We obtained participant-level data of the PROMISE trial through the National Heart, Lung and Blood Institute. Details of the PROMISE trial have been previously published. Briefly, PROMISE (ClinicalTrials.gov identifier: NCT01174550) recruited 10,003 patients from multiple centers in the USA and Canada who were randomized to either anatomical (CTCA) or functional testing (including exercise electrocardiography, nuclear stress testing, or stress echocardiography). We confirm that the present study complied with the Declaration of Helsinki.

### Study Population and Covariates

In PROMISE, we identified all individuals who underwent initial assessment with anatomical or functional testing, consistent with their original randomized assignment. This represented 9,572 of the 10,003 original participants. We included patient characteristics available at trial enrollment, including demographics (age, sex, race, ethnicity), anthropometrics [body mass index (BMI)], cardiovascular risk factors (systolic and diastolic blood pressure, hypertension, diabetes mellitus, smoking status, family history), laboratory measurements (haemoglobin, creatinine, lipid panel), medications, presenting symptoms (i.e. chest pain, shortness of breath), chest pain characteristics (typical, atypical, non-cardiac), electrocardiographic parameters (e.g., rhythm, Q waves, findings interfering with stress test interpretation), and clinical risk scores (pooled cohort equation derived 10-year atherosclerotic cardiovascular disease risk and modified Diamond-Forrester risk for obstructive coronary artery disease). We excluded variables from model development if they were missing in over half of the participants or if they were recorded after study initiation. We imputed missing data for the included variables using chained random forests with predictive mean matching. Following imputation, we transformed continuous variables into standardized scores (z-scores) by subtracting their mean and dividing by their respective standard deviation

### Study Outcomes

To ensure consistency with the original trials, we used each study prespecified primary endpoint. In PROMISE, our primary study population, we trained our models using a composite of death, myocardial infarction (MI), unstable angina hospitalization, or major procedural complication (major adverse cardiovascular events [MACE]). We also identified a secondary composite endpoint of all-cause mortality and non-fatal MI.

### Defining Phenotypic Neighborhoods

In PROMISE, we computed a dissimilarity index that classified individuals based on 57 pre-randomization characteristics according to the Gower distance, a metric of dissimilarity between two patients based on mixed numeric and non-numeric data. For continuous variables, Gower distance represents the absolute value of the difference between a pair of individuals divided by the range across all individuals. For categorical variables the method assigns “1” if the values are identical and “0” if they are not. Gower distance is ultimately calculated as the mean of these terms. Alternatively, the dissimilarity index can be computed based on cosine similarity, or other similarity measures. For each patient in PROMISE, we identified a topological neighborhood of the 5% most phenotypically similar participants based on Gower's distance. In sensitivity analyses, we iteratively evaluated random neighborhood sizes between 2.5% and 10%, assessing the correlation of effect estimates in these iterations with those derived from the 5% neighborhood size.

### Individualized Risk Phenomapping

Within each patient-centered neighborhood, we assessed the association of undergoing anatomical vs. functional imaging with MACE in age- and sex-adjusted Cox regression models, thus providing individualized risk estimates based on each patient's unique neighborhood. The natural logarithmic transformations of the hazard ratio (HR) from the Cox models comparing anatomical and functional testing for each patient's topological neighborhood represented their individualized effect estimate. In our approach, negative log-HRs favor anatomical testing, whereas positive values favor functional imaging. In an alternative embodiment of this approach, a weighted Cox regression model can be fitted for each original trial participant, with unique weights assigned to each original trial participant based on their similarity to the index patient of each neighborhood. This enables iterative analyses of the original clinical trial by applying a unique kernel to the original observations based on each patient's unique phenotype.

Furthermore, since an unbiased personalized effect estimate is contingent upon the similarity of individuals in their topological neighborhoods, we created a measure of neighborhood homogeneity. This represented the square of 1 minus the average pairwise distance between the index patient and each one of their neighbors, with higher values reflecting a neighborhood of phenotypically more similar patients.

To visualize the phenotypic variation in the PROMISE population and neighborhoods we used uniform manifold approximation and projection (UMAP), which constructs a two-dimensional representation of the high-dimensional feature space. We employed color maps to visualize the topological distribution of the patient baseline demographics and neighborhood estimates in the phenomap.

We demonstrated the ability of our approach to detect heterogeneity in treatment effects using examples of individuals sharing a key set of features (age, sex, traditional risk factors) but differing on other baseline characteristics.

### Extreme Gradient Boosting Algorithm to Predict the Benefit of Anatomical Testing

To translate the heterogeneity in treatment effect across the PROMISE phenomap to a clinical population, we constructed an extreme gradient boosting algorithm to predict the personalized risk of MACE with anatomical vs. functional imaging (natural logarithm of the neighborhood HR) using routinely collected variables, which were available in >50% of participants, spanning demographics, comorbidities, laboratory testing, vitals, and medications with implications for anatomical or functional testing. We included 21 variables including key demographics (age, sex), risk factors (smoking, family history of CAD, hypertension, diabetes mellitus, total cholesterol, high-density lipoprotein, statin use), anthropometrics (BMI, systolic and diastolic blood pressure), cerebrovascular and peripheral vascular disease, ECG findings (rhythm, Q waves, findings interfering with stress test interpretation, as defined in PROMISE), and use of antiplatelets and beta-blockers.

We randomly divided the PROMISE population into training (80%, n=7660) and validation (20%, n=1912) sets. Briefly, we trained the extreme gradient boosting algorithm to identify patient characteristics that were strongly associated with improved outcomes (patient-centered log-hazards) for anatomical or functional testing. We used root mean squared error to evaluate our model performance, identified the optimal hyperparameters using a grid search, and implemented 10-fold cross-validation. We evaluated feature importance using SHAP (SHapley Additive exPlanations) values, which identify a predictor contribution, either positively or negatively, to the prediction.

To improve the model practical application, we selected features that were strongly associated with improved outcomes with either anatomical or functional testing based on a feature importance of 0.03 or higher, resulting in 12 features. We retrained our model using these limited set of features, using 10-fold cross-validation in the 80% of PROMISE, followed by further validation in the remaining (unseen) 20% of PROMISE.

This machine-learning-derived parsimonious model trained on 12 features represented ASSIST (Anatomical vs. Stress teSting decIsion Support Tool). Negative ASSIST values (<0) favored functional-first assessment.

### Statistical Analyses

We compared the two treatment groups using Student's t-test for continuous variables and chi-square test for categorical variables and used Pearson's correlation to assess continuous variables. We performed survival analyses using Cox proportional-hazards regression. While neighborhoods were matched on pre-randomization covariates, we explicitly adjusted Cox models for age and sex. We assessed the association of the ASSIST recommended testing modality and outcomes through its groupwise interactions with the two treatment groups in Cox models. Statistical tests were two-sided with a level of significance of 0.05. Analyses were performed using R (version 4.0.2) and Python (version 3.8.5).

Results

### Study Population

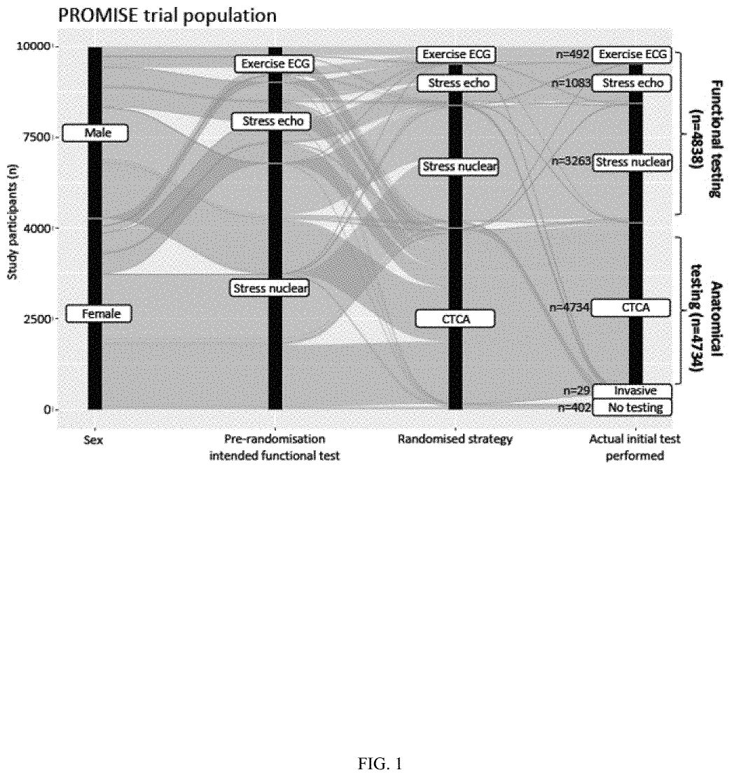

From PROMISE, we included 9,572 patients [age 60.3±8.3 years, n=5013 (52.4%) women] with stable chest pain. Of these, 4,734 (49.5%) underwent CTCA and the remaining 4,838 (50.5%) functional imaging (FIG. 1A). Baseline characteristics were balanced between the two study arms. Over a mean follow-up period of 2.1±0.9 years, there were 294 MACE (primary study outcome), with no significant difference in the primary outcome in the two arms [adjusted HR 1.03 (95% confidence interval (CI): 0.82-1.29), P=0.8159 for anatomical vs. functional imaging].

### Phenomapping the Stable Chest Pain in PROMISE

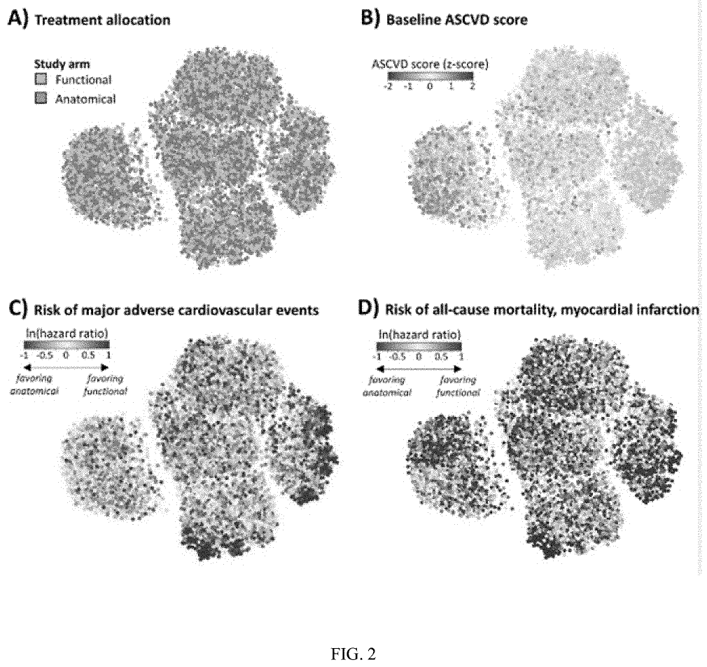

We first created a phenomap of our study population using a pairwise dissimilarity metric derived from 57 pre-randomization phenotypic characteristics and visualized it as a two-dimensional manifold representation. Based on visual assessment, the two treatment arms were distributed uniformly throughout the phenotypic space, consistent with their random allocation across the population (FIG. 2A) with varying baseline clinical factors and risk of CAD (FIG. 2B).

### Distribution of Neighborhood-Based Individualized Risk Estimates

Patient-specific neighborhoods for each of the 9572 included PROMISE participants, included 5% of the population in their topological vicinity, with a wide distribution of neighborhood-specific risk effect estimates. The median neighborhood-specific HR for MACE was 1.11 with 10th, 25th, 75th, and 90th percentiles of 0.52, 0.76, 1.67, and 2.61, respectively. A projection of each person's individual effect estimate on the phenomap suggested distinct topological neighborhoods favoring anatomical or functional testing (FIGS. 2C and D). There was also variation in both the direction of the effect and the effect size for different endpoints across the topological space of the study population.

In sensitivity analyses for variable neighborhood sizes (2.5%, 5%, 7.5%, 10%, 15% of the study population), an increasing neighborhood size was associated with a narrower distribution of individual risk estimates around the average treatment effect across the cohort (HR 1.03), representing loss of risk heterogeneity observed at larger neighborhood sizes. The larger neighborhood, however, also compared dissimilar individuals with decreasing neighborhood homogeneity based on increasing mean distances. Random iterations for various neighborhood sizes between 2.5% and 10% showed that the average effect size was strongly correlated with that derived from 5% neighborhoods [r=0.72 (95% CI 0.71-0.73)].

### Using Risk Phenomap for Individualized Risk Prediction

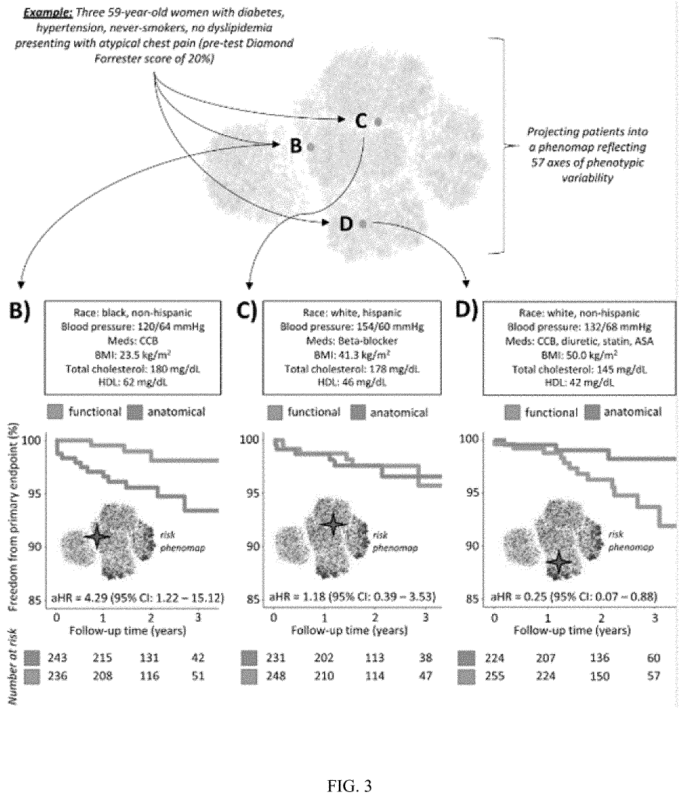

To demonstrate an example of individualized risk estimation using the phenomap, we identified a subset of three phenotypically similar PROMISE participants, each of them a 59-year-old woman, with a history of diabetes and hypertension but not smoking, presenting with atypical chest pain and a modified pre-test Diamond-Forrester score of 20%. Despite the above similarities, phenomapping using all 57 included variables revealed that these patients were located in distinct topological neighborhoods (FIG. 3A). Each patient's neighborhood-specific assessments identified differential risk/benefit associated with anatomical vs. functional testing, ranging from improved outcomes with functional imaging (FIG. 3B) to similar outcomes with either strategy (FIG. 3C), or improved outcomes with anatomical imaging (FIG. 3D). Of note, each patient neighborhood had phenotypically similar patients in the two study arms.

The Anatomical vs. Stress Testing Secision Support Tool (ASSIST)

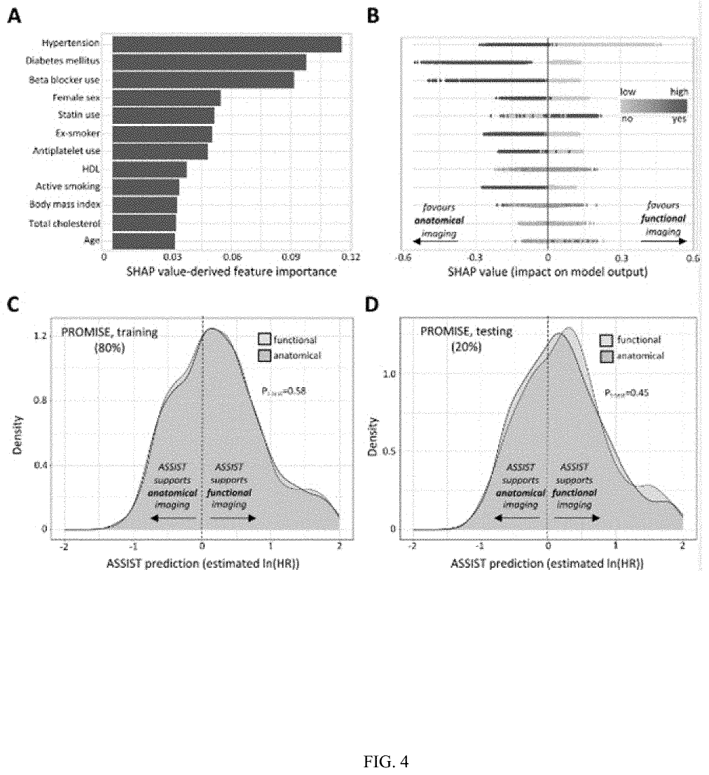

In the 80% training set from PROMISE (n=7660), an extreme gradient boosting algorithm identified hypertension, diabetes mellitus, use of beta-blockers, female sex, statin use, smoking history, antiplatelet use, BMI, age, and cholesterol levels as the predictors with highest feature importance for relative hazard of MACE with anatomic or functional testing (FIG. 4A). Feature importance analysis suggested that female sex, hypertension, diabetes mellitus, use of beta-blockers, and active or former smoking were each associated with improved outcomes with anatomical testing (FIG. 4B), whereas absence of these risk factors as well as lower BMI and statin use favored functional testing. Our clinical decision support tool, ASSIST, represents the extreme gradient model developed using these 12 most important features. Hold-out validation performance of the parsimonious 12-feature tool was comparable with that of a model relying on all 21 inputs (RMSE of 0.59 vs. 0.57, respectively), while logistically easier to deploy.

Of note, in both the cross-validated training and testing sets of PROMISE, there was no association between the ASSIST risk prediction and the allocation to either anatomical or functional imaging, consistent with the random allocation to the two arms (FIGS. 4C and D).\

### Validation of ASSIST

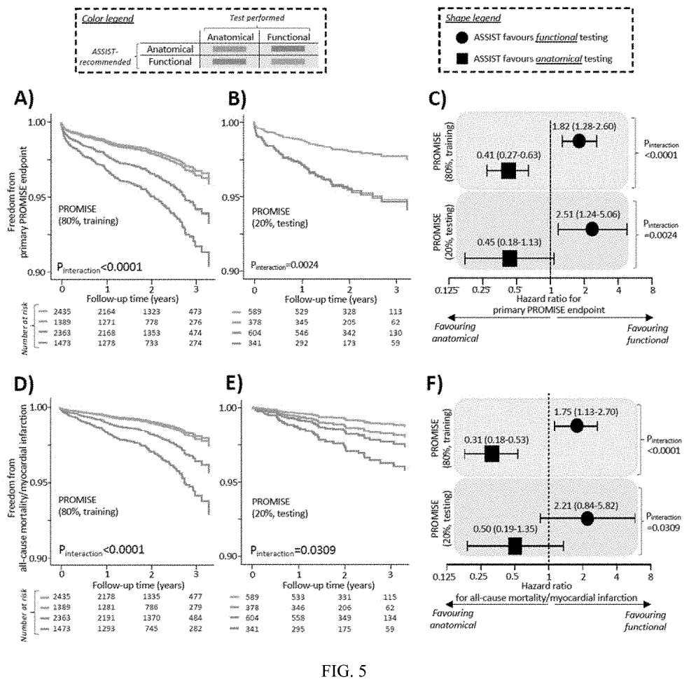

In the remaining 20% PROMISE participants (n=1912, validation; FIG. 5), the ASSIST performed well in identifying the favored diagnostic strategy. An agreement between the ASSIST recommendation (score >0: favoring functional, score <0: favoring anatomical) and the actual test per-formed was associated with a significantly lower incidence of each primary composite endpoint (FIGS. 5A-C) as well as the endpoint of all-cause mortality and non-fatal MI (FIGS. 5D-F) with consistent significant interaction between the ASSIST-recommended test and performed strategy (FIG. 5).

Discussion

In the largest clinical trial to have evaluated the role of CTCA in the investigation of stable chest pain, we developed and validated a ma-chine learning-based decision support tool to guide the selection between anatomical and functional evaluation. We defined a novel strategy that constructs a high-dimensional phenotypic representation of trial participants, permitting a series of local experiments with-in the trial uncovering heterogeneous treatment effects, identifying individuals who may derive benefit from one strategy over another. Our approach synthesizes the complex relationship between a large number of pre-randomization characteristics in creating and visualizing a comprehensive phenomap of patients, with an individualized assessment of the risk of adverse cardiovascular events with anatomical or functional imaging for assessing chest pain. Our new machine-learning-derived tool (ASSIST) based on 12 widely available clinical parameters derived from risk phenomaps reliably and consistently identified patients who were more likely to have improved outcomes when assigned to an anatomical or functional diagnostic strategy. To date, there has been no consensus on the strategy to choose between anatomical and functional testing in chest pain evaluation, and different clinical practice guidelines provide varying levels and strengths of recommendation on the use of CTCA vs. functional testing. Despite PROMISE, identifying a population that may benefit from CTCA or functional imaging has been mostly supported through post hoc analyses in large population sub-groups, specifically women, and patients with diabetes, and considerations about CTCA test characteristics, including high sensitivity, but limited specificity in detecting haemodynamically significant lesions. Therefore, the default strategy may be to use CTCA in individuals at presumably low-to-intermediate risk of CAD. Unfortunately, this approach does not benefit from the knowledge gained from the large clinical trials and the extensive phenotypic variability among trial participants. Our approach overcomes these limitations through a specific focus on a large feature set and their complex relationship to each other, therefore deriving a personalized estimate, as opposed to an average treatment effect across large heterogeneous groups. In addition, instead of focusing on the absolute risk of obstructive disease or myocardial ischaemia, our study explores the factors associated with the relative benefit obtained from anatomical vs. functional testing.

Our study uses a novel approach to achieve these goals. Our approach leverages the detailed phenotypic characterization of clinical trial populations at enrolment and the unbiased treatment allocation to infer a personalized treatment effect. Therefore, it provides a quantitative evaluation of the heterogeneity of out-comes, and an assessment whether the average treatment effect observed in a clinical trial setting applies to a given trial participant. We also created a visual representation of differences across individuals enrolled in a clinical trial, allowing interpretability of different patients and the observed effects. Our approach builds upon prior studies that have employed clustering to demonstrate clinical trial participants have discordant effects. However, they are limited in clinical application as they ultimately represent broad subgroups of patients that differ from each other on many characteristics, thereby limiting a personalized treatment selection. In our approach, each individual represents the center of their own cluster and, therefore, is compared with similar individuals in inferring a treatment effect.

In addition, our machine-learning-based decision support tool, ASSIST, allows such personalization of the diagnostic strategy for chest pain using only 12 key variables. The tool consistently demonstrated a lower rate of all-cause mortality and ad-verse cardiovascular outcomes where the diagnostic strategy was aligned with ASSIST recommendation.

Conclusion

We have developed an approach that defines an evidence-based strategy to pursue anatomical or functional evaluation of patients with suspected CAD. The approach uses a series of local experiments in a multidimensional phenomap of trial participants to infer a personalized strategy of the diagnostic evaluation approach most likely to achieve the best outcomes. Furthermore, a generalizable decision support tool derived from this phenomap, and validated in a large clinical, enables a broader use of this in-formation in shared decision-making in clinical practice.

### Experiment 2

We subsequently evaluated the performance of the methods across multiple different domains, and across clinical trials of several therapeutic agents. An example of this application is the application of phenomapping to the CANVAS trials. The CANVAS trials include CANVAS, a study that randomized patients with type 2 diabetes and elevated cardiovascular risk to receiving canagliflozin or placebo in 2:1 ratio on the background of other diabetes therapies, followed for adverse cardiovascular events. A second study, the CANVAS-R, included patients randomized 1:1 to canagliflozin and placebo. Phenomapping applied to the CANVAS trial identified heterogeneity in the effect of canagliflozin across the phenotypic spectrum of the trial, and was used to create a tool INSIGHT, similar to ASSIST, that defined an individual's cardiovascular benefit from canagliflozin using a set of baseline characteristics. This is an important observation because of the cost of canagliflozin. The tool INSIGHT was externally validated in the CANVAS-R trial that was completely independent of the derivation trial, CANVAS, and identified individuals in CANVAS-R that derived most benefit from the use of canagliflozin. We also find that a small number of individuals with defined phenotypic characteristics defined a majority of the benefit observed in the trial, with implications for efficient trial design.

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method for phenotype mapping clinical trial participants, the method comprising:
receiving a set of data corresponding to a plurality of characteristics for a plurality of individual participants;
classifying each individual patient based on the plurality of characteristics and according to a dissimilarity index;
determining a dissimilarity value for each individual patient with respect to each of the remaining individual patients; and
generating a phenotype neighborhood map comprising graphical representations for each individual patient, wherein a distance between one individual patient and another individual patient is according to the dissimilarity value determined for the one patient with respect to the other individual patient.

2. The method of claim 1, further comprising:
grouping each individual patient into a neighborhood based on a phenotype similarity threshold and the determined dissimilarity values.

3. The method of claim 1, wherein the plurality of characteristics comprise demographics, anthropometrics, health condition risk factors, laboratory measurements, medications, health condition symptoms, clinical risk scores, imaging or other medical data, or a combination thereof.

4. The method of claim 1, further comprising:
identifying a treatment to be administered to the plurality of individual patients;
selecting a set of characteristics from the plurality of characteristics; and
determining a heterogeneity level in effects from the treatment on a subset of individual patients sharing the selected set of characteristics.

5. The method of claim 4, further comprising:
identifying a plurality of characteristics for an individual apart from the individual trial participants; and
determining a treatment outcome for the administered treatment and for the individual based on the determined heterogeneity level.

6. The method of claim 4, wherein the treatment to be administered comprises a medication, procedural or surgical intervention, nutritional supplement, diagnostic or therapeutic strategy, or a combination thereof.

7. The method of claim 1, further comprising:
identifying a treatment to be administered to the plurality of individual patients; and
training a machine-learning algorithm to identify associations above a predefined threshold between one or more of the plurality of characteristics and a patient result of the administered treatment.

8. The method of claim 7, wherein the machine-learning algorithm is an extreme gradient boosting algorithm.

9. The method of claim 7, further comprising:
retraining the machine-learning algorithm by selecting a different set of characteristics; and
identifying associations between the different set of characteristics and the patient result of the administered treatment.

10. The method of claim 1, wherein the phenotype neighborhood map comprises graphical representations.
